(19) United States
(12) Patent Application Publication
(10) Pub. No.: US 2009/0275980 A1
(43) Pub. Date: Nov. 5, 2009

(54) METHOD AND APPARATUS FOR THE FORMATION OF TISSUE FOLDS

(76) Inventors: Mark S. Zeiner, Mason, OH (US); Michael J. Stokes, Cincinnati, OH (US); Jason L. Harris, Mason, OH (US); Wayne Young, Brewster, NY (US); James T. Spivey, Cincinnati, OH (US)

Correspondence Address: WELSH & FLAXMAN LLC, 2000 DUKE STREET, SUITE 100, ALEXANDRIA, VA, 22314, US

(21) Appl. No.: 12/113,633

(22) Filed: May 1, 2008

(51) Int. Cl. A61B 17/04

(52) U.S. Cl. 606/232

(57) ABSTRACT

A suture anchoring device includes a fastener body composed of a tube and a spool is mounted within the tube for rotation relative to the tube. Suture material is wrapped about the spool such that pulling of the suture material causes rotation of the spool within the tube. A one-way locking mechanism allows the spool to rotate freely in a first direction and prevents rotation in an opposite direction. A method for creating a tissue fold includes deploying a first suture anchoring device within the tissue with suture material extending proximally from the first suture anchoring device, deploying a second suture anchoring device within the tissue with a distal portion of the suture material extending between the first suture anchoring device and a proximal portion of the suture material extending proximally from the second suture anchoring device, and applying tension to the suture material to draw portions of the tissue together to form a tissue fold.

### BACKGROUND OF THE INVENTION

1. Field of the Invention

The invention relates to bariatric surgery. More particularly, the invention relates to a method and medical apparatus for the creation of folds in the reduction of gastric volume.

2. Description of the Related Art

Obesity is a medical condition affecting more than 30% of the population in the United States. Obesity affects an individual's personal quality of life and contributes significantly to morbidity and mortality. Obese patients, i.e., individuals having a body mass index (“BMI”) greater than 30, often have a high risk of associated health problems (e.g., diabetes, hypertension and respiratory insufficiency), including early death. With this in mind, and as those skilled in the art will certainly appreciate, the monetary and physical costs associated with obesity are substantial. In fact, it is estimated the costs relating to obesity are in excess of 100 billion dollars in the United States alone. Studies have shown that conservative treatment with diet and exercise alone may be ineffective for reducing excess body weight in many patients. Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In a RYGB procedure a small stomach pouch is separated from the remainder of the gastric cavity and attached to a resectioned portion of the small intestine. This resectioned portion of the small intestine is connected between the “smaller” gastric cavity and a distal section of small intestine allowing the passage of food therebetween. The conventional RYGB procedure requires a great deal of operative time. Because of the degree of invasiveness, post-operative recovery can be quite lengthy and painful. Still more than 100,000 RYGB procedures are performed annually in the United States alone, costing significant health care dollars.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing a feeling of satiety. A band is placed around the stomach near the junction of the stomach and esophagus. The small upper stomach pouch is filled quickly, and slowly empties through the narrow outlet to produce the feeling of satiety. Other forms of bariatric surgery that have been developed to treat obesity include Fobi pouch, bilio-pancreatic diversion and gastroplasty or “stomach stapling”.

Morbid obesity is defined as being greater than 100 pounds over one's ideal body weight. For individuals in this category, RYGB, gastric banding or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss.

It is known to create cavity wall plications through endoscopic only procedures. However, operating solely within the interior of the gastric cavity limits the plication depth that can be achieved without cutting. Furthermore, access and visibility within the gastric and peritoneal cavities is limited in a purely endoscopic procedure as the extent of the reduction increases.

With the foregoing in mind, it is desirable to have a surgical weight loss procedure that is inexpensive, with few potential complications, and that provides patients with a weight loss benefit while buying time for the lifestyle changes necessary to maintain the weight loss. Further, it is desirable that the procedure be minimally invasive to the patient, allowing for a quick recovery and less scarring. The present invention provides such a procedure.

### SUMMARY OF THE INVENTION

It is, therefore, an object of the present invention to provide a suture anchoring device including a fastener body including a tube and a spool is mounted within the tube for rotation relative to the tube. Suture material is wrapped about the spool such that pulling of the suture material causes rotation of the spool within the tube. A one-way locking mechanism allows the spool to rotate freely in a first direction and preventing rotation in an opposite direction.

It is also an object of the present invention to provide a suture anchoring device wherein the tube includes a slot providing access of the suture material to the spool.

It is another object of the present invention to provide a suture anchoring device wherein the suture anchoring device is a T-tag fastener.

It is a further object of the present invention to provide a suture anchoring device wherein the one-way locking mechanism includes internal threading formed within the tube shaped and dimensioned for engagement with external threading formed on the spool.

It is also an object of the present invention to provide a suture anchoring device wherein the one-way locking mechanism further includes a biasing spring biasing the external threading formed on the spool toward the internal threading formed within the tube.

It is another object of the present invention to provide a method for creating a tissue fold including deploying a first suture anchoring device within the tissue with suture material extending proximally from the first suture anchoring device, deploying a second suture anchoring device within the tissue with a distal portion of the suture material extending between the first suture anchoring device and a proximal portion of the suture material extending proximally from the second suture anchoring device, and applying tension the suture material to draw portions of the tissue together to form a tissue fold.

It is a further object of the present invention to provide a suture anchoring device where the second suture anchoring device includes a one-way locking mechanism.

It is also an object of the present invention to provide a suture anchoring device wherein the one-way locking mechanism allows the suture material to be drawn in one direction and prevents movement of the suture material in the second direction opposite the first direction.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1 to 7 show the steps in deploying first and second T-tag fasteners along the gastric wall for the formation of a serosa-to-serosa fold.

FIG. 8 is a detailed view of the second T-tag fastener in accordance with present invention.

### DESCRIPTION OF THE PREFERRED EMBODIMENTS

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as a basis for teaching one skilled in the art how to make and/or use the invention.

With reference to the various figures, tags, fasteners and anchors are disclosed for the formation of folds in the performance of gastric reduction surgery. The tags, fasteners and anchors disclosed herein can, however, be used for multiple applications in various surgeries. However, the present disclosure will focus upon their usage in the formation of serosa-to-serosa plications in the gastric antrum created during gastric reduction surgery.

As disclosed in commonly owned U.S. patent application Ser. No. 11/779,322, entitled “HYBRID ENDOSCOPIC/LAPAROSCOPIC METHOD FOR FORMING SEROSA TO SEROSA PLICATIONS IN A GASTRIC CAVITY”, filed Jul. 18, 2007, which is incorporated herein by reference, serosa-to-serosa plications are utilized in the reduction of gastric cavity volume during bariatric procedures aimed at assisting patients with weight loss. Briefly, available stomach volume may be restricted by forming one or more folds in the anterior wall 16 of the gastric cavity 10 (creating a serosa-to-serosa fold 18). The folds 18 reduce the outer surface area of the gastric cavity 10 and, correspondingly, the available food volume within the gastric cavity 10. In accordance with one restriction technique, the available volume within the gastric cavity 10 is restricted by forming a single, longitudinally extending fold 18 along the anterior wall 16 of the gastric cavity 10. The fold 18 extends the full length of the anterior wall 16 of the gastric cavity 10 between the fundus and the pylorus. Alternatively, a shorter fold may be formed depending upon the desired amount of gastric volume reduction.

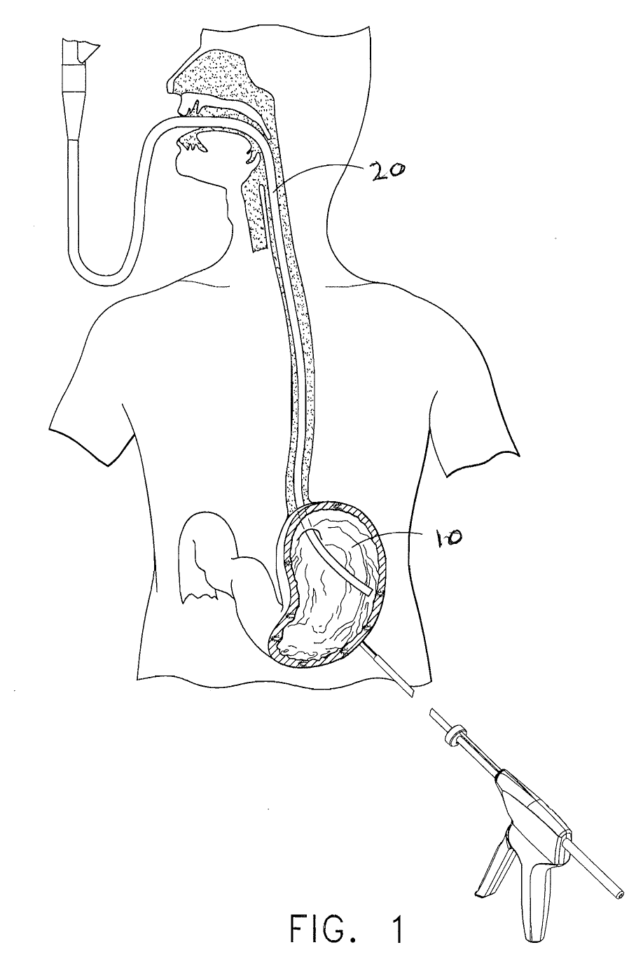

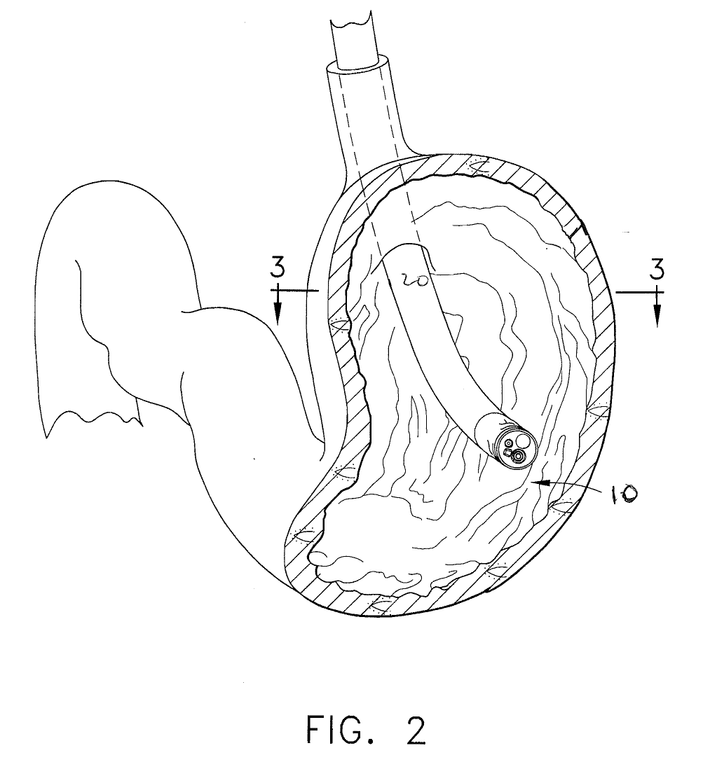

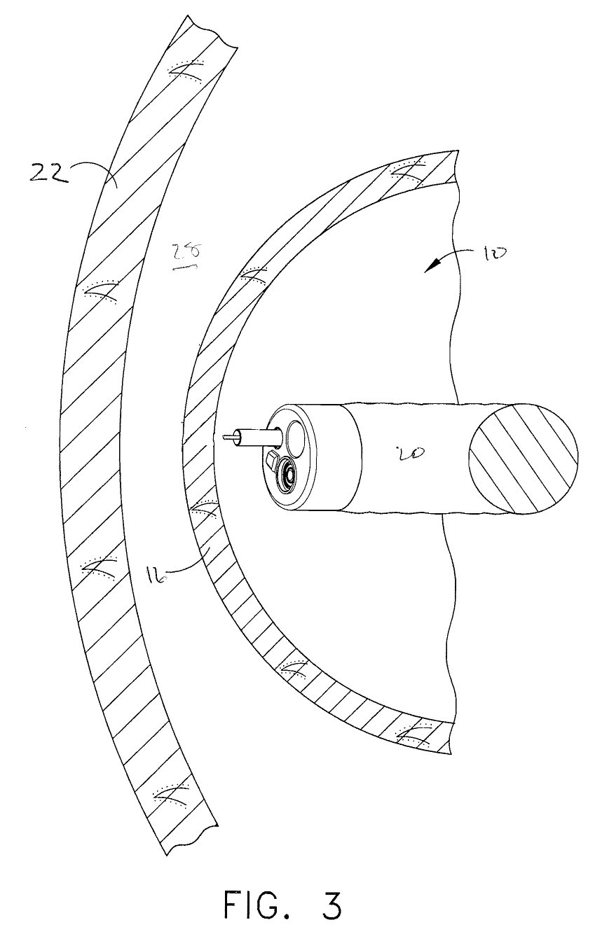

Generally, to form a fold in accordance with the present invention, a flexible gastroscope 20 is passed transesophageally into the gastric cavity 10 as shown in FIGS. 1, 2 and 3. The gastroscope 20 provides insufflation, illumination and visualization of the gastric cavity 10, as well as a passageway into the gastric cavity 10 for the insertion and use of other endoscopic instruments. The gastric cavity 10 is first insufflated to create a sufficient rigid working surface along the gastric cavity 10 such that it may be pierced without damaging the opposing wall of the gastric cavity 10. Insufflation of the gastric cavity 10 may also allow the boundaries of the gastric cavity 10 and the desired location for a fold 18 to be mapped out by external palpation. The pressure on the abdominal wall 22 is observed within the gastric cavity 10 through the gastroscope 20 to also determine the appropriate placement of one or more trocars (or other ports allowing abdominal access) for completion of the procedure in accordance with the present invention.

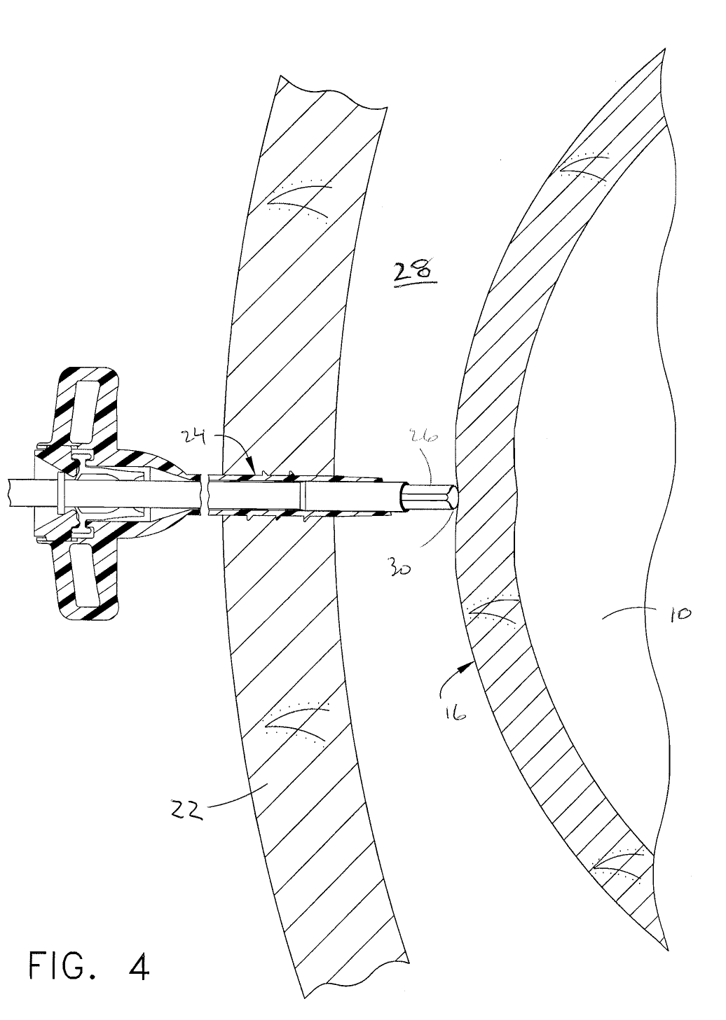

After the gastric cavity 10 has been mapped through the gastroscope 20, a trocar 24 is inserted through the abdominal wall 22. FIG. 4 shows a trocar 24 inserted through the abdominal wall 22 allowing access to the exterior of the gastric cavity 10. The placement of the trocar 24 depends upon the intended location of the fold 18, in particular, the serosa-to-serosa fold. It should be noted that with insufflation of the peritoneal cavity the trocar could be inserted in the same locations as are typically used for gastric banding or RYGB procedures (that is, not directly above the stomach). The trocar 24 preferably has a diameter of between approximately 3 mm and approximately 5 mm to allow an adequate sized passageway for instruments and suture anchoring devices, that is, T-tag fasteners 12 in accordance with a preferred embodiment of the present invention.

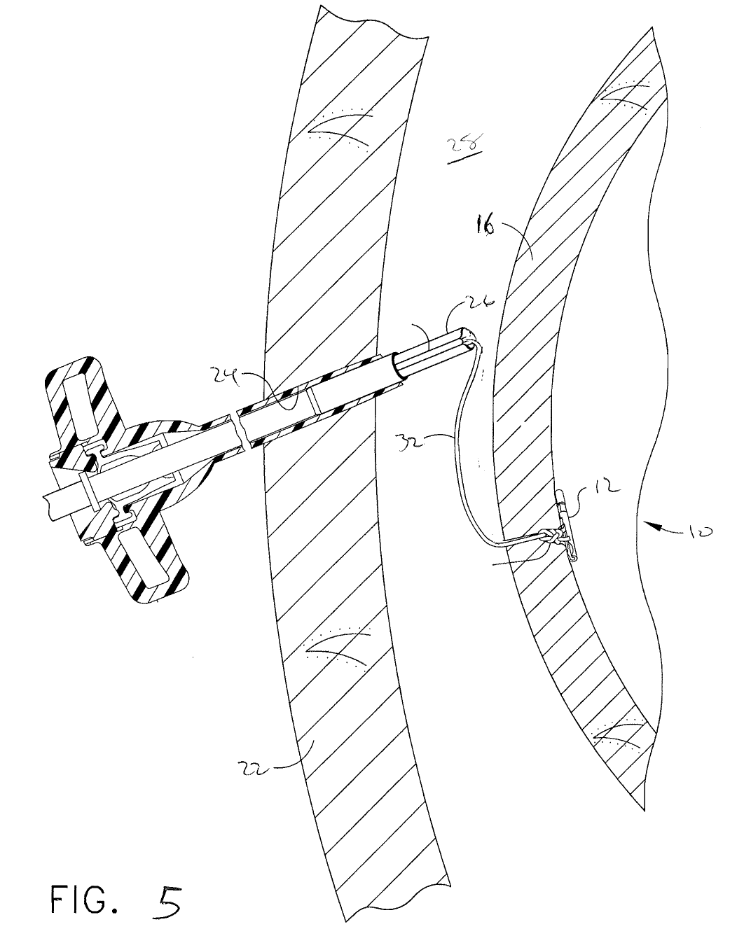

With the trocar inserted into the abdominal wall 22, a suture anchor deployment device 26 is passed through the trocar 24 into the abdominal cavity 28. Prior to insertion of the deployment device 26, the tip 30 of the deployment device 26 is pressed against the anterior wall 16 of the gastric cavity 10 to indent the wall, as shown in FIG. 4. The indentation along the anterior wall 16 of the gastric cavity 10 is visualized through the gastroscope 20 (from within the gastric cavity) to determine the proper location to insert the deployment device 26 into the gastric cavity 10. After the proper insertion location is determined, the tip 30 of the deployment device 26 is inserted through the anterior wall 16 and into the interior of the gastric cavity 10. The deployment device 26 is inserted into the gastric cavity 10 with sufficient force to prevent the deployment device 26 from glancing off of the exterior surface of the anterior wall 16 of the gastric cavity 10. After the tip 30 of the deployment device 26 is inside the gastric cavity 10, a conventional suture anchoring device, in particular, a first T-tag fastener 12 is deployed from the deployment device 26 into the interior of the gastric cavity 10 with the suture material 32 from the T-tag fastener 12 fixedly attached to the T-tag and extending proximally therefrom (see FIG. 5).

While a conventional T-tag fastener is disclosed for use in accordance with a preferred embodiment of the present invention, other suture anchoring devices may be utilized within the spirit of the present invention. Examples of suitable tissue fasteners include t-type anchors as already discussed, reconfigurable “basket”-type anchors (which generally comprise a number of configurable structure legs extending between two collars or support members), and linear anchors (elongated anchors which are configured to fold or become compressed into a bowed or expanded configuration). In general, anchor characteristics are such that prior to deployment, they can easily be placed into or through tissue(s), but after deployment, have an altered configuration providing at least one dimension sufficiently large to maintain the anchor in place. It is also envisioned that fasteners used in this location need not completely penetrate the gastric wall to the interior of the gastric cavity.

After the T-tag fastener 12 is deployed into the gastric cavity 10, the deployment device 26 is removed from the gastric cavity 10. As the deployment device 26 is removed, the suture material 32 attached at the distal end to the T-tag fastener 12 extends from the T-tag fastener 12 and through the anterior wall 16 of the gastric cavity 10. The proximal end 34 of the suture material 32 extends through the trocar 24 and outside the body.

After the deployment device 26 is removed from the anterior wall 16 of the gastric cavity 10, the anterior wall 16 again is probed with the tip 30 of the deployment device 26 to determine the location for another suture anchoring device, in particular, second T-tag fastener 112. To facilitate the probing of the anterior wall 16, the trocar 24 may be flexed at different angles within the abdominal wall 22 without removing the trocar 24 from the abdominal wall 22. The trocar 24 is angled within the abdominal wall 22 to enable the deployment device 26 to enter the gastric cavity 10 at different locations and in a different direction roughly perpendicular to the exterior surface of the gastric cavity 10. If the angles are such that perpendicular to the exterior surface is not achievable then a grasper can be used through another trocar port to bring the anterior wall tissue up to allow a perpendicular angle to the tissue. Once the proper placement location is determined, the deployment device 26 is once again inserted into the gastric cavity 10. With the deployment device 26 inside the gastric cavity 10, the second T-tag fastener 112 is deployed into the interior of the gastric cavity 10 with the distal portion of the suture material 32 extending between the first T-tag fastener 12 and the proximal portion of the suture material extending proximally from the second T-tag fastener 112.

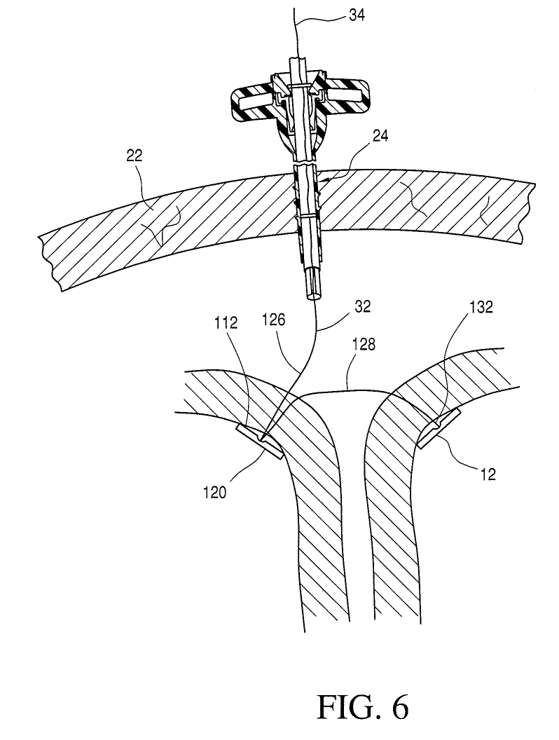

After the second T-tag fastener 112 is deployed, the deployment device 26 is removed from the gastric cavity 10, drawing the length of suture material 32 back through the anterior wall 16 of the gastric cavity 10. The proximal end 34 of the length of suture material 32 is drawn through the trocar 24 and external of the body. As shown with reference to FIG. 6, the suture material extends with its proximal end external to the body and its central portion in contact with the second T-tag fastener 112 for movement relative thereto and its distal end secured to the first T-tag fastener 12.

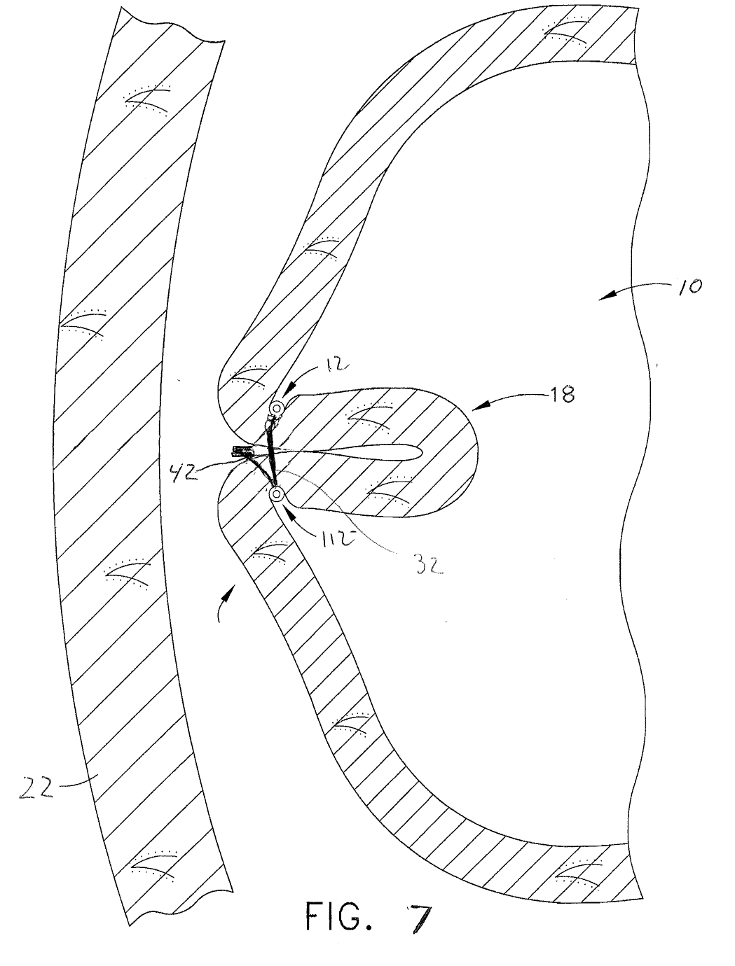

Tension is then applied to the proximal end of suture material to draw the fastened portions of the anterior wall 16 of the gastric cavity 10 together to form a serosa-to-serosa fold 18 as shown in FIG. 7. Because the suture material 32 is fixedly connected to the first T-tag fastener 12 and connected to the second T-tag fastener 112 for movement relative thereto, when tension is applied to the suture material 32 the suture material 32 is drawn proximally with additional suture material being drawn beyond the second T-tag fastener 112 and external to the body. In order to ensure that suture material 32 drawn proximally relative to the second T-tag fastener 112 does not move in the opposite direction (that is, increasing the length of the suture material between the first and second T-tag fasteners 12, 112 and allowing the tissue being drawn in apposition to move apart), the second T-tag fastener 112 is provided with a one-way locking mechanism 120 allowing the suture material 32 to be drawn in one direction (that is, proximally in accordance with a preferred embodiment of the present invention) and prevent movement of the suture material in the second direction (that is, distally in accordance with a preferred embodiment of the present invention).

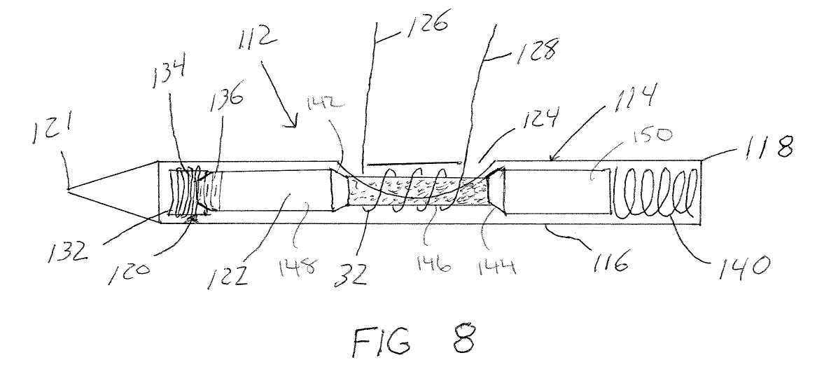

Referring to FIG. 8, the second T-tag fastener 112 is shown in detail. The second T-tag fastener 112 includes a fastener body 114 composed of a tube 116 including a closed first end 118 and a closed second end 121. A spool 122 is mounted within the tube 116 for rotation relative thereto and within the tube 116. As will be discussed below in greater detail, the suture material 32 is wrapped about the spool 122 such that pulling of the suture material 32 causes rotation of the spool 122 within the tube 116.

With this in mind, and in order to allow access of the suture material 32 to the spool 122, the tube 116 is provided with a slot 124 having two openings which separate the leading end 126 and the trailing end 128 of the suture material 32 as the suture material respectively extends toward the proximal end 34 of the suture material 32 and the distal end 132 of the suture material 32 secured to the first T-tag fastener 12.

Controlled rotation of the spool 122, and therefore controlled movement of the suture material 32 relative thereto is achieved by a one-way locking mechanism 120 composed of a spring 140 biasing the spool 122 toward the second end 121 of the tube 116 where the second end 121 of the tube 116 is provided with internal threading 134 for engagement with external threading 136 formed on the second end 138 of the spool 122. As such, when suture material 32 is drawn in the second direction, that is, distally toward the first T-tag fastener 12, the external threading 136 along the second end 138 of the spool 122 and the second end 121 of the tube 116 interlock and reach a stopping point which prevents further rotation of the spool 122 and further movement of the suture material 32 in a distal direction. Seating of the external threading 136 at the second end 138 of the spool 122 with the internal threading 134 at the second end 121 of the tube 116 is ensured by the provision of a biasing spring 140 that forces the spool 122 toward the second end 121 of the tube 116. However, when the spool 122 is rotated in the opposite direction, that is, the direction as the suture material 32 is drawn in a first direction proximally external to the body, the external threading 136 at the second end 138 of the spool 122 merely rides over the internal threading 134 of the second end 121 of the tube 116 (much in the manner a bottle cap rotates over threading when rotated in a reverse direction) and the spool 122 may be rotated indefinitely in this direction since the threading along the second end 121, 138 of the spool 122 and tube 116 never engage and lock.

By utilizing a second T-tag fastener 112 such as disclosed in accordance with the present invention, reverse motion of the opposed tissue positions is prevented because the suture material 32 is regularly locked in position as the threading 134, 136 along the second ends 121, 138 of the spool 122 and tube 116 engage when rotated in the second direction. In addition, because the spool 122 may rotate indefinitely so long as it is rotated in the first direction based upon proximal pulling of the suture material 32, the distance and placement between the first and second T-tags fasteners 12, 112 is irrelevant.

It is contemplated the second T-tag may achieve one-way rotation through the utilization of a ratchet mechanism or through the deformation of the tubular member based upon rotation thereof. It is further contemplated that prevention of the suture material from slipping upon the spool may be achieved by wrapping the suture material at least three times about the central portion of the spool so as to create a capstan effect thereon that will eliminate slippage thereof. In addition, slippage may be prevented by increasing the frictional interaction between the suture material and the spool. The suture may rotate the shaft smoothly without binding by providing a shaft shaped to specifically eliminate binding and provide separate entrances and exits for the suture to help prevent binding. As shown with reference to FIG. 8, and in accordance with a preferred embodiment, the spool 122 includes transition zones 142, 144 between the central section 146 on which the suture material 32 is spooled and the outer sections 148, 150 of the spool 122 which fit inside the tube 116, wherein the central section 146 has a small diameter than outer sections 148, 150. These transition zones 142, 144 preferably assume a concave shape. This shape serves to pull the suture material 32 towards the mid-portion of the central section 146 as the spool 122 is rotated, preventing the suture material 32 from bunching or wedging its way into the gap between the spool 122 and the tube 116.

Once the suture material 32 is fully drawn proximally and the tissue fold along the anterior wall of the gastric cavity is completely formed at the location of the first and second T-tag fasteners 12, 112, a knotting element 42 may be applied to the proximal end of the length of the suture material 32 to hold the tissue together in its apposed orientation. In addition to knotting elements, the suture material may also be locked in a tensioned state by tying a knot in the suture material. The knot may be tied laparoscopically through the trocar. Alternatively, the knot may be tied external of the body, and the finished knot passes back through the trocar to a point between the abdominal wall and the anterior wall of the gastric cavity.

After the first pair of T-tag fasteners 12, 112 is deployed, the trocar 24 and the deployment device 26 may be angled within the abdominal wall 22 to again probe the gastric cavity 10 and determine additional locations for the application of additional pairs of T-tag fasteners. The additional pairs of T-tag fastener are preferably spaced down the length of the anterior wall 16 from the first pair of T-tag fasteners 12, 112 in order to extend the length of the fold 18. Once the location for an additional pair of the T-tag fasteners is determined, the deployment device 26 is again inserted through the anterior wall 16 of the gastric cavity 10 to deploy a the T-tag fasteners in manner described above with regard to the first pair of T-tag fasteners.

While applying the additional pair of the T-tag fasteners, the trocar may be flexed within the abdominal wall, or removed and repositioned within the abdominal wall as necessary, in order to reach all of the desired T-tag fastener locations. The suture material is cinched together between each pair of the suture anchoring devices to extend the cavity wall fold. The number of T-tag fasteners used to form a fold will depend upon the desired length of the fold (and also the depth of the fold or number of consecutive rows of fasteners). Preferably, each of the pairs of the T-tag fasteners is evenly spaced apart along the length of the anterior wall of the gastric cavity. Likewise, each individual pair of T-tag fasteners is evenly spaced apart across the fold line, so that a uniform tissue fold is formed without distortion or bunching. The proper relative spacing of the T-tag fasteners can be ascertained through the gastroscope. Alternatively, an additional trocar may be inserted into the abdominal wall and used in conjunction with an optical instrument to visually determine the proper locations for the T-tag fasteners.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used system is obtained and if necessary cleaned. The system can then be sterilized. In one sterilization technique, the system is placed in a closed and sealed container, such as a plastic or TYVEK bag. The container and system are then placed in a field of radiation that can penetrate the container, such as gamma radiation, x-rays, or high-energy electrons. The radiation kills bacteria on the system and in the container. The sterilized system can then be stored in the sterile container. The sealed container keeps the system sterile until it is opened in the medical facility.

It is preferred that the device is sterilized. This can be done by any number of ways known to those skilled in the art including beta or gamma radiation, ethylene oxide and/or steam.

While the preferred embodiments have been shown and described, it will be understood that there is no intent to limit the invention by such disclosure, but rather, is intended to cover all modifications and alternate constructions falling within the spirit and scope of the invention.

1. A suture anchoring device, comprising:
a fastener body including a tube and a spool is mounted within the tube for rotation relative to the tube;
suture material is wrapped about the spool such that pulling of the suture material causes rotation of the spool within the tube; and
a one-way locking mechanism allowing the spool to rotate freely in a first direction and preventing rotation in an opposite direction.

2. The suture anchoring device according to claim 1, wherein the tube includes a slot providing access of the suture material to the spool.

3. The suture anchoring device according to claim 1, wherein the suture anchoring device is a T-tag fastener.

4. The suture anchoring device according to claim 1, wherein the one-way locking mechanism includes internal threading formed within the tube shaped and dimensioned for engagement with external threading formed on the spool.

5. The suture anchoring device according to claim 4, wherein the one-way locking mechanism further includes a biasing spring biasing the external threading formed on the spool toward the internal threading formed within the tube.

6. A method for applying suture anchoring devices to tissue in creation of a tissue fold, comprising:
deploying a first suture anchoring device within the tissue with suture material extending proximally from the first suture anchoring device;
deploying a second suture anchoring device within the tissue with a distal portion of the suture material extending between the first suture anchoring device and a proximal portion of the suture material extending proximally from the second suture anchoring device; and
applying tension to the suture material to draw portions of the tissue together to form a tissue fold.

7. The method according to claim 6, where the second suture anchoring device includes a one-way locking mechanism.

8. The method according to claim 7, wherein the one-way locking mechanism allows the suture material to be drawn in a first direction and prevents movement of the suture material in a second direction opposite the first direction.

9. The method according to claim 6, wherein the second suture anchoring device includes a fastener body composed of a tube and a spool is mounted within the tube for rotation relative to the tube, the suture material being wrapped about the spool such that pulling of the suture material causes rotation of the spool within the tube; and a one-way locking mechanism allowing the spool to rotate freely in a first direction and preventing rotation in an opposite second direction.

10. The method according to claim 9, wherein the tube includes a slot providing access of the suture material to the spool.

11. The method according to claim 9, wherein the second suture anchoring device is a T-tag fastener.

12. The method according to claim 9, wherein the one-way locking mechanism includes internal threading formed within the tube shaped and dimensioned for engagement with external threading formed on the spool.

13. The method according to claim 12, wherein the one-way locking mechanism further includes a spring biasing the external threading formed on the spool toward the internal threading formed within the tube.
